(19) United States
(12) Patent Application Publication
(10) Pub. No.: US 2023/0380999 A1
(43) Pub. Date: Nov. 30, 2023

(54) STENT AND TREATMENT SYSTEM WITH SUCH A STENT

(71) Applicant: Acandis GmbH, Pforzheim (DE)

(72) Inventors: Michael Buchert, Pforzheim (DE); Alexander Lange, Karlsruhe (DE)

(21) Appl. No.: 18/202,716

(22) Filed: May 26, 2023

(30) Foreign Application Priority Data
May 27, 2022 (CN) 2022105932859
May 27, 2022 (DE) 102022113422.9

(51) Int. Cl. A61F 2/90; A61F 2/958

(52) U.S. Cl. CPC A61F 2/90; A61F 2/958; A61F 2230/0017; A61F 2230/0028

(57) ABSTRACT

The disclosure relates to a stent with a compressible and expandable mesh structure of webs which are interconnected by web connectors into one piece and define rhomboid cells, wherein each cell is defined by two straight webs and two S-shaped curved webs which connect the straight webs together, and wherein (i) in a non-operational state, the mesh structure has a fully expanded non-operational diameter Dexp which is between 3.0 mm and 5.0 mm, (ii) a ratio between a fully compressed diameter Dkomp of the mesh structure and the non-operational diameter Dexp of the mesh structure is between 1:7 and 1:12, and (iii) the webs have a web height, measured in a radial direction, which is at least 0.05 mm and at most 0.09 mm, so that the mesh structure has a radial force of at least 0.5 N, in particular at least 0.6 N, between the fully compressed diameter Dkomp and an operational diameter which is at most 90% of the non-operational diameter Dexp.

### CROSS-REFERENCE TO RELATED APPLICATIONS

The present application is a U.S. non-provisional patent application that claims priority to both Chinese Patent Application No. 2022105932859 filed May 27, 2022 and German Patent Application No. 102022113422.9, filed May 27, 2022. Each of these applications are hereby incorporated by reference herein in their entirety.

### TECHNICAL FIELD

The invention relates to a stent and a treatment system.

### BACKGROUND

The treatment of arteriosclerotic intracranial arterial stenosis (ICAS) usually comprises two treatment steps. In a first treatment step, the stenosis is dilated by means of a balloon catheter and in a second treatment step, a stent is placed in the area of the stenosis. This treatment method is extremely complicated, especially as it requires the use of a balloon catheter on the one hand and a delivery catheter for the stent on the other. This necessitates a long time in surgery, with increased risks for the patient.

A particular difficulty lies in treating stenoses which have formed in small vessels, especially in the neurovascular field. This requires stents which have a very small delivery profile in order to be capable of being guided through the small blood vessels to the treatment site. Stents of this type must therefore be capable of being compressed to a very small cross-sectional diameter. However, in practice, stents which can be compressed to such a small cross-sectional diameter have a lower radial force, so until now, they have not been able to be used for the treatment of stenoses. Rather, such stents are in fact known, but are used for the treatment of aneurysms.

### SUMMARY

The objective of the invention is to provide a stent which has been improved on the basis of the known prior art and which permits particularly good treatment of stenoses, especially in very small blood vessels. In particular, the stent exhibits a high radial force with a small compressed cross-sectional diameter. Furthermore, the objective of the invention is to provide a treatment system with such a stent.

Thus, the invention is based on the concept of providing a stent with a compressible and expandable mesh structure of webs which are interconnected by means of web connectors into one piece and define rhomboid cells, wherein each cell is delimited by two straight webs and two S-shaped curved webs which connect the straight webs together, and wherein
in a non-operational state, the mesh structure has a fully expanded non-operational diameter which is between 3.0 mm and 5.0 mm,
a ratio between a fully compressed diameter of the mesh structure and the non-operational diameter of the mesh structure is between 1:7 and 1:12,
the webs have a web height, measured in a radial direction, which is at least 0.05 mm and at most 0.09 mm, so that the mesh structure has a radial force of at least 0.5 N, in particular at least 0.6 N, between the fully compressed diameter and an operational diameter which is at most 90% of the non-operational diameter.

The invention therefore provides a stent which, because of the large ratio between the fully compressed diameter and the non-operational diameter of the mesh structure, can readily be introduced into small blood vessels and at the same time, because of its increased web height, applies a sufficient radial force to treat a stenosis in a sustainable manner. In this context, it should be pointed out that the stent is dimensioned in a manner such that the mesh structure has a non-operational diameter which is between 3.0 mm and 5.0 mm in a fully expanded state. The non-operational diameter here corresponds to the diameter of the mesh structure in the fully expanded state without the influence of external forces.

In general, the mesh structure can be expanded from a compressed diameter to an expanded diameter and vice versa. In this context, the “compressed diameter” is the diameter at which the mesh structure has its smallest cross-sectional diameter. The mesh structure is preferably self-expandable.

In a preferred embodiment, the straight webs have a first web width and the S-shaped curved webs have a second web width, wherein the first web width is at least 25% and at most 33% larger than the second web width. The different web widths provide the mesh structure with a particularly high flexibility, which makes the mesh structure ideal for introduction into small, in particular very tortuous blood vessels. Such blood vessels are present in particular in the cerebral vessel region. In this respect, the invention is particularly suitable for the treatment of stenoses in the intracerebral region.

The web connectors of a cell which are consecutively aligned in a longitudinal direction of the mesh structure can have a spacing in the non-operational state which defines a cell length, wherein the cell length is between 2.0 mm and 3.6 mm. The limited cell length of the cells of the mesh structure has a positive effect on what is known as foreshortening. Foreshortening is the process by which the mesh structure shortens axially as a result of its radial expansion. This foreshortening makes positioning of the mesh structure in the blood vessel more difficult and should therefore be as small as possible. This is achieved with the cell lengths given here.

It is possible for the web connectors of a cell which are consecutively aligned in the circumferential direction of the mesh structure to have a spacing in the non-operational state which defines a cell height, wherein the cell height is between 1.5 mm and 2.7 mm. It has been shown that such a cell height offers a good compromise between a mesh structure which is as flexible as possible and good coverage of the vessel surface covered by the mesh structure. Good coverage is particularly advantageous with regard to the radial force and the associated treatment of stenoses. At the same time, the mesh structure should remain flexible in the compressed state so that it can navigate well through blood vessels. The cell heights described here contribute significantly to enabling this dual function.

In connection with the cell length described here, the cell height is of particular importance. Depending on the respective non-operational diameter of the mesh structure, the ratio between cell length and cell height contributes to improved handling of the stent. In particular, the foreshortening on the one hand and the radial force on the other hand as well as the flexibility of the mesh structure during delivery are positively influenced by a predetermined ratio between cell height and cell length. In this respect, it is preferable for the ratio between the cell length and the cell height to be between 1.3 and 1.41.

A simplification of the treatment of intracerebral stenoses results in particular from the combination of the stent described above with a catheter in the context of a treatment system which will be described below. In fact, the treatment system in accordance with the invention makes it possible to dilate the stenosis using a balloon catheter and deliver the stent for the subsequent support of the stenosis with a single catheter. Additional treatment steps are therefore avoided. This leads to a reduction in surgery time and to a lower risk for the patient.

More specifically, an ancillary aspect of the invention concerns a treatment system for the medical treatment of intracranial stenoses comprising a stent as described above and a catheter for delivering the stent into a hollow organ of the body, wherein the catheter has at least two working channels and a balloon disposed in the distal region of the working channels, wherein a first working channel is in fluid communication with the balloon and a second working channel extends through the balloon, wherein the second working channel is a through channel with an inner diameter of at most 0.44 mm.

The second working channel with an internal diameter of at most 0.44 mm (0.17 inch or 2.0 French), in particular at most 0.42 mm (0.165 inch or 1.9 French), enables small stents as described in the present application to be delivered. This makes the catheter particularly suitable for the treatment of stenoses in intracerebral vessels. The combination with the stent described herein is particularly advantageous because, despite its small size, the stent offers a relatively high radial force, which is a prerequisite for the stent to be suitable for the treatment of stenoses. A particular innovation of the treatment system is that the second working channel of the catheter has a wall which is made up of four layers, at least in sections. Preferably, a first, innermost layer contains polytetrafluoroethylene (PTFE). The first, innermost layer is preferably coated with a second layer of a stabilizing braid. The stabilizing braid can be formed by a mesh structure of a superelastic material, for example a nickel-titanium alloy, or from a stainless steel. Furthermore, the second layer may be surrounded by a third layer of a polyimide (PI). Finally, a fourth, outermost layer may also be provided which is formed from a polyether block amide (PEBA) and surrounds the third layer. In particular, the innermost layer of polytetrafluoroethylene has particularly friction-reducing properties, so that delivery of the stent, which provides increased radial force, is facilitated. The second layer, of a stabilizing braid, supports the catheter and in particular prevents ovalisation of the catheter. At the same time, the second layer provides support in the axial direction so that the catheter can be advanced easily.

### BRIEF DESCRIPTION OF THE DRAWINGS

The invention will explained in more detail below with the aid of an example of an embodiment with reference to the accompanying diagrammatic drawings, in which

FIG. 1 shows a developed view of a mesh structure of the stent in accordance with the invention according to a preferred exemplary embodiment;

FIG. 2 shows a cross-sectional view of the stent according to FIG. 1 in a fully compressed state;

FIG. 3 shows a cross-sectional view of the stent according to FIG. 1 in a fully expanded state;

FIG. 4 shows a top view of a section of the developed mesh structure in accordance with FIG. 1, wherein dimensions of the webs and cells are shown;

FIG. 5 shows a longitudinal sectional view through a catheter of the treatment system in accordance with the invention according to a preferred embodiment; and

FIG. 6 shows a cross-sectional view through the first working channel of the catheter according to FIG. 5.

### DESCRIPTION

The stent in accordance with the invention has a mesh structure 10 which is formed from a plurality of webs 11. The webs 11 are interconnected by means of web connectors 14 into one piece. Specifically, the mesh structure 10 may be cut from a tube.

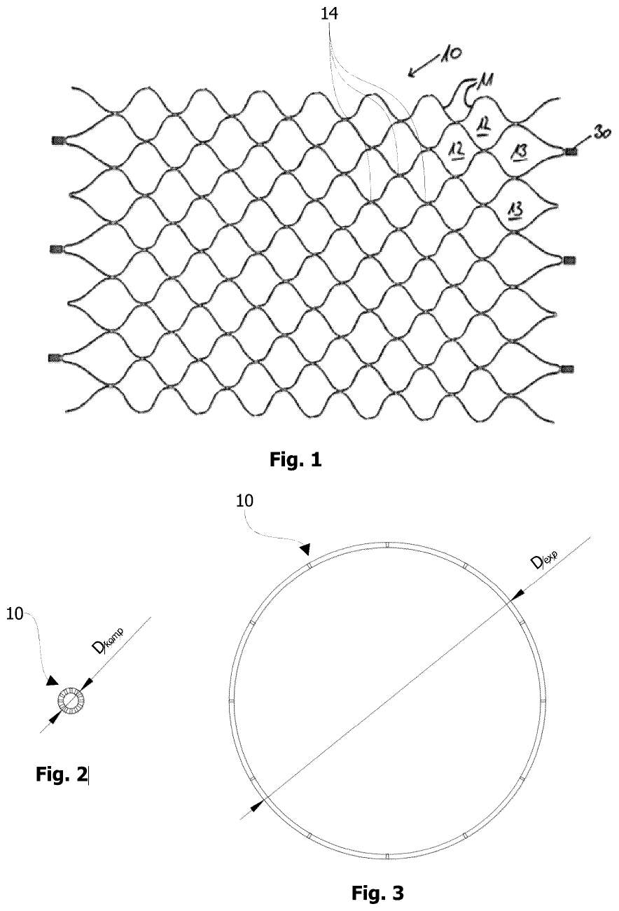

FIG. 1 shows the mesh structure 10 in a developed state, wherein the mesh structure 10 has been cut open along its longitudinal side and spread out. The webs 11, which delimit the cells 12, are also a non-operational state, as are the cells 12, i.e. unaffected by external forces. This state is also referred to as the fully expanded state.

As can also be seen in FIG. 1, the shape of the cells 12, which have an essentially rhomboid basic form, deviates in the peripheral regions of the mesh structure 10. Specifically, end cells 13 are provided at the axial ends of the mesh structure 10 which have a slightly different geometry than the cells 12. Each second end cell 13 also carries a radiographic marker 30. The radiographic marker 30 may comprise a radiopaque material, for example platinum, in particular platinum-iridium, or gold. Preferably, the radiographic marker 30 is formed as a sleeve which is crimped onto a web extension of the end cell 13.

The mesh structure 10 is preferably formed from a self-expanding material. Such a self-expanding or superelastic material is a shape memory material, for example. Preferably, a nickel-titanium alloy is used for this purpose.

The geometry of the stent in accordance with the invention combines two essential advantages. On the one hand, the stent in accordance with the invention provides a good radial force which is particularly suitable for the treatment of stenoses in blood vessels. At the same time, the mesh structure 10 is so flexible that it can be expanded from a very small compressed diameter to a comparatively large, preferably 12-fold larger, expanded non-operational diameter.

The relationship between the compressed diameter Dₖₒₘₚ and the expanded non-operational diameter Dₑₓₚ is clearly visible in FIGS. 2 and 3. In this regard, FIG. 2 shows the mesh structure 10 in a fully compressed state, wherein the mesh structure has the compressed diameter Dₖₒₘₚ. FIG. 3, on the other hand, shows the fully expanded state of the mesh structure 10, wherein no external forces act on the mesh structure 10. The fully expanded state thus corresponds to a non-operational state. In this case, the mesh structure 10 has a non-operational diameter Dₑₓₚ. The ratio between the fully compressed diameter Dₖₒₘₚ and the non-operational diameter Dₑₓₚ is preferably between 1:7 and 1:12. This means that the mesh structure has a non-operational diameter Dₑₓₚ of between 3.0 mm and 5.0 mm.

The operational diameter of the mesh structure 10 must be distinguished from the non-operational diameter (nominal diameter). The geometry of the stent means that the radial force for supporting a stenosis is particularly sufficient when the mesh structure 10 is expanded to a diameter which is at most 90% of the non-operational diameter Dₑₓₚ. It is particularly preferred if the operational diameter is at most 90% of the non-operational diameter Dₑₓₚ. With such an operational diameter, the radial force of the stent or the mesh structure is preferably at least 0.5 Newton, in particular at least 0.6 Newton. Radial force values of this type have been shown to be particularly efficient for the treatment of stenoses.

In order to obtain such radial force values, in the stent in accordance with the invention, the web height, which is measured in the radial direction, i.e. along the cross-sectional diameter shown in FIGS. 2 and 3, is provided between 0.05 mm and 0.09 mm. Such a web height on the one hand enables good compression of the mesh structure 10 to be obtained on a very small compressed diameter Dₖₒₘₚ, and on the other hand provides a radial force which is sufficiently large to keep a stenosis in a blood vessel permanently open.

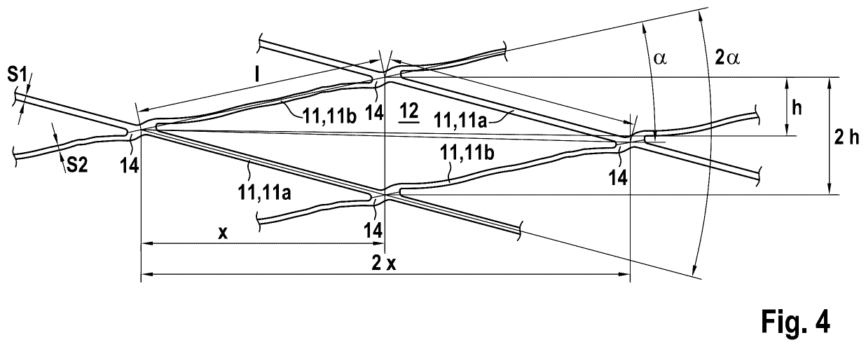

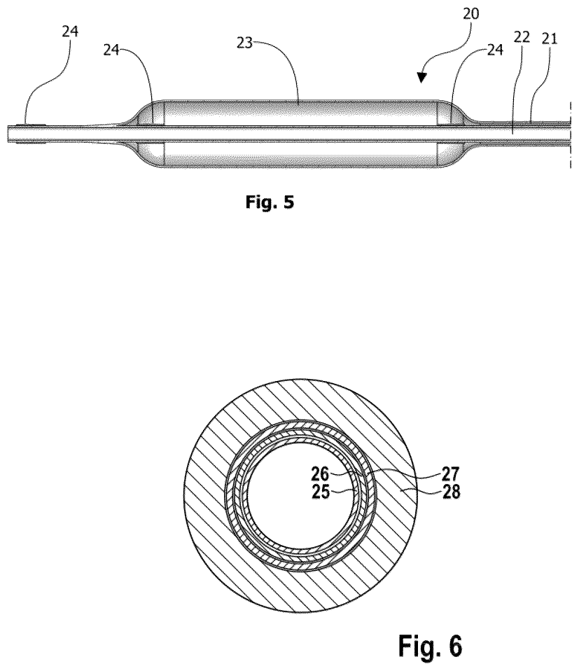

The geometry of the stent according to the embodiments shown here is illustrated in FIG. 4. Specifically, FIG. 4 shows a section of a mesh structure 10, wherein a cell 12 is shown in its entirety. In FIG. 4, the cell 12 is in a state after the mesh structure 10 has been cut from a raw material (“as-cut”). The raw material is preferably a tube with a diameter of 1.5 mm, i.e. the mesh structure 10 has a diameter of 1.5 mm in the state according to FIG. 4. The mesh structure 10 is then expanded to the non-operational diameter and the shape memory material is packaged at the non-operational diameter. In this manner, different mesh structures 10 with different non-operational diameters can be formed from the raw material with a diameter of 1.5 mm.

The cell 12 is delimited by the webs 11, wherein two straight webs 11a are arranged parallel to each other and are interconnected by S-shaped curved webs 11b. The connection between the straight webs 11a and the curved webs 11b is made via web connectors 14 in each case. Each web connector 14 couples together a total of four webs 11, namely two straight webs 11a and two curved webs 11b.

For the advantageous properties of the stent in accordance with the invention, it is necessary to adjust individual parameters of the stent geometry. These parameters are in particular the web height, in particular the web widths S1, S2, the cell length 2x, the cell height 2h and the cell angle 2α.

Thus, the straight webs 11a have a first web width S1 and the curved webs 11b have a second web width S2. The first web width S1 is larger here than the second web width S2. In particular, the first web width S1 can be at least 25% and at most 33% larger than the second web width S2.

The cell length 2x is defined by the distance between two web connectors 14, which are directly consecutively aligned with each other in the longitudinal direction of the mesh structure 10. The cell length 2x is preferably between 2.0 mm and 3.6 mm.

The distance between two web connectors 14 which are directly adjacent and aligned with each other in the circumferential direction corresponds to the cell height 2h and is preferably between 1.5 mm and 2.7 mm.

Overall, it has been shown to be particularly advantageous for the ratio between the cell length 2x and the cell height 2h to be between 1.3 and 1.41.

In a variation in accordance with the invention, the stent described here is preferably used with a modified catheter 20 as the treatment system. The treatment system therefore comprises the stent and the catheter 20, wherein these two elements are matched to each other. In particular, the increased radial force provided by the stent is supported by the constructional design of the catheter 20.

The catheter 20 generally has a first working channel 21 and a second working channel 22. The first working channel 21 is in fluid communication with a balloon 23. A fluid, in particular a liquid, can therefore be introduced into the balloon 23 via the first working channel 21 and removed from it again. The balloon 23 can therefore be filled or inflated and expanded or deflated via the first working channel 21.

The second working channel 22 is preferably arranged coaxially in the first working channel 21. The second working channel 22 extends through the balloon 23 and forms a distal tip of the catheter 20. A marker ring 24 is preferably provided at the distal tip of the catheter 20. Two marker rings 24 are also provided on the second working channel 22 inside the balloon 23. The marker rings 24 preferably mark the longitudinal ends of the balloon 23, so that the longitudinal extent of the balloon 23 can be detected under radiographic monitoring.

The structure of the wall of the second working channel 22 is particularly decisive for good delivery of the stent described above with the increased radial force. The second working channel 22 has a wall formed by four layers. A first, innermost layer 25 preferably has friction-reducing properties. In particular, the first layer 25 may comprise or be formed from polytetrafluoroethylene (PTFE), which reduces the frictional forces between a stent and the second working channel 22.

The second working channel 22 is designed as a through channel so that the stent described above can be advanced from a proximal end of the catheter 20 directly along to a distal end of the catheter 20. This allows the stent to be implanted without having to remove the catheter 20 from the blood vessel beforehand. The catheter 20 with the distal balloon 23 thus enables a combined treatment method to be carried out. In a first step of the treatment, a stenosis can be radially dilated via the catheter and its balloon 23. Without having to remove or change the catheter 20, it is then possible to insert a stent into the stenosis, which is then expanded to keep the stenosis permanently open.

The first layer 25 of polytetrafluoroethylene is encased by a second layer 26. The second layer 26 of the second working channel 22 preferably comprises a stabilizing braid. The stabilizing braid may be formed from a metal or a metal alloy. Preferably, a nickel-titanium alloy or a stainless steel is used. In each case, the stabilizing braid protects the second working channel 22 from becoming ovalised, i.e. from deviating from a circular cross-sectional shape, when being turned through blood vessels with different curvatures. In addition, the stabilizing braid stabilizes the entire catheter 20 in the longitudinal direction, which facilitates advancement of the catheter 20 into a blood vessel.

The second layer 26 is sheathed by a third layer 27, which preferably consists of polyimide. A fourth layer 28, which forms the outer sheath of the second working channel 22, is preferably formed of a polyether block amide (PEBA). As FIG. 6 clearly shows in cross-section, the first layer 25, the second layer 26 and the third layer 27 have a substantially similar or identical wall thickness. The fourth, outermost layer 28, however, has a wall thickness which is larger than the wall thickness of the other three layers 25, 26, 27 together.

### REFERENCE NUMERALS

10 mesh structure
11 web
11 a straight web
11b curved web
12 cell
13 end cell
14 web connector
20 catheter
21 first working channel
22 second working channel
23 balloon
24 marker ring
25 first, innermost layer
26 second layer
27 third layer
28 fourth, outermost layer
30 radiographic marker
Dₖₒₘₚ compressed diameter
Dₑₓₚ expanded non-operational diameter
S1 first web width
S2 second web width
h half cell height
2h cell height
x half cell length
2x cell length
α half cell angle
2α cell angle

1-8. (canceled)

9. A stent comprising:
a compressible and expandable mesh structure of webs which are interconnected by web connectors into one piece and define rhomboid cells, wherein each cell is defined by two straight webs and two S-shaped curved webs which connect the straight webs together, and wherein,
in a non-operational state, the mesh structure has a fully expanded non-operational diameter between 3.0 mm and 5.0 mm,
a ratio between a fully compressed diameter of the mesh structure and the non-operational diameter of the mesh structure is between 1:7 and 1:12, and
the webs have a web height, measured in a radial direction, which is at least 0.05 mm and at most 0.09 mm, so that the mesh structure has a radial force of at least 0.5 N between the fully compressed diameter and an operational diameter which is at most 90% of the non-operational diameter.

10. The stent according to claim 9, wherein the straight webs have a first web width and the S-shaped curved webs have a second web width, and wherein the first web width is at least 25% and at most 33% larger than the second web width.

11. The stent according to claim 9, wherein the web connectors of a cell are consecutively aligned in a longitudinal direction of the mesh structure and have spacing in the non-operational state which defines a cell length between 2.0 mm and 3.6 mm.

12. The stent according to claim 11, wherein the web connectors of the cell are consecutively aligned in a circumferential direction of the mesh structure and have spacing in the non-operational state which defines a cell height between 1.5 mm and 2.7 mm.

13. The stent according to claim 12, wherein a ratio of the cell length to the cell height is between 1.3 and 1.41.

14. A treatment system for medical treatment of intracranial stenoses comprising:
a stent having a compressible and expandable mesh structure of webs which are interconnected by web connectors into one piece and define rhomboid cells, each cell defined by two straight webs and two S-shaped curved webs which connect the straight webs together, and
a catheter for delivering the stent into a hollow organ of a body,
wherein the catheter has at least two working channels and a balloon disposed in a distal region of the working channels,
wherein a first working channel is in fluid communication with the balloon and a second working channel extends through the balloon, and
wherein the second working channel is a through channel having an inner diameter of at most 0.44 mm.

15. The treatment system according to claim 14, wherein the second working channel has a wall constructed from four layers in sections.

16. The treatment system according to claim 15, wherein a first, innermost layer comprises polytetrafluoroethylene and is coated with a second layer of a stabilizing braid, wherein the second layer is surrounded by a third layer of a polyimide and wherein the third layer is surrounded by a fourth, outermost layer of a polyether block amide (PEBA).

17. A stent comprising:
a compressible and expandable mesh structure of webs which are interconnected by web connectors into one piece and define rhomboid cells, wherein each cell is defined by two straight webs and two S-shaped curved webs which connect the straight webs together, wherein the straight webs have a first web width and the S-shaped curved webs have a second web width such that the first web width is at least 25% larger than the second web width, and wherein,
in a non-operational state, the mesh structure has a fully expanded non-operational diameter between 3.0 mm and 5.0 mm,
a ratio between a fully compressed diameter of the mesh structure and the non-operational diameter of the mesh structure is between 1:7 and 1:12, and
the webs have a web height, measured in a radial direction, of at least 0.05 mm and at most 0.09 mm, so that the mesh structure has a radial force of at least 0.5 N between the fully compressed diameter and an operational diameter which is at most 90% of the non-operational diameter.

18. The stent according to claim 17, wherein the web connectors of a cell are consecutively aligned (i) in a circumferential direction of the mesh structure with spacing in the non-operational state which defines a cell height between 1.5 mm and 2.7 mm, and (ii) in a longitudinal direction of the mesh structure with spacing in the non-operational state which defines a cell length between 2.0 mm and 3.6 mm.

19. The stent according to claim 18, wherein a ratio of the cell length to the cell height is between 1.3 and 1.41.

20. The stent according to claim 17, wherein the web connectors of a cell are consecutively aligned in a longitudinal direction of the mesh structure and have spacing in the non-operational state which defines a cell length between 2.0 mm and 3.6 mm.

21. The stent according to claim 17, wherein the web connectors of a cell are consecutively aligned in a circumferential direction of the mesh structure and have spacing in the non-operational state which defines a cell height between 1.5 mm and 2.7 mm.
